(19) United States
(12) Patent Application Publication
(10) Pub. No.: US 2015/0147707 A1
(43) Pub. Date: May 28, 2015

(54) CANDLE SCENT DEVICE

(71) Applicant: Jay D. Hatfield, Fort Wayne, IN (US)

(72) Inventor: Jay D. Hatfield, Fort Wayne, IN (US)

(21) Appl. No.: 14/549,973

(22) Filed: Nov. 21, 2014

Related U.S. Application Data
(60) Provisional application No. 61/907,416, filed on Nov. 22, 2013.

(51) Int. Cl. A61L 9/03

(52) U.S. Cl. CPC A61L 9/037
USPC 431/320; 431/289

(57) ABSTRACT

A candle including a wick, a combustible material in contact with a portion of the wick and a scent container. The scent container has an elongated body configured to hold a fragrant liquid. The scent container being configured to generally surround the wick.

### CROSS REFERENCE TO RELATED APPLICATIONS

This is a non-provisional application based upon U.S. provisional patent application Ser. No. 61/907,416, entitled “CANDLE SCENT DEVICE”, filed Nov. 22, 2013, which is incorporated herein by reference.

### BACKGROUND OF THE INVENTION

1. Field of the Invention

The present invention relates to an oil candle, and, more particularly, to scent releasing device associated with an oil candle.

2. Description of the Related Art

Traditionally a candle is a block of wax with an embedded wick which is ignited to provide light, and in some instances heat. A candle manufacturer is traditionally known as a chandler. Various devices have been used to display candles, from simple tabletop candle holders, to elaborate chandeliers.

For a candle to burn, a flame is used to light the wick of the candle, the heat therefrom melts and vaporizes a small amount of the wax. Once vaporized, the now liquid wax combines with oxygen in the atmosphere to ignite and form a constant flame. This flame provides sufficient heat to keep the candle burning by way of a self-sustaining set of events: the heat of the flame melts the a portion of the wax; the liquefied wax then moves upward through the wick by capillary action; and the liquefied wax vaporizes to burn within the candle's flame.

As the wax is melted and consumed, the candle grows shorter. Portions of the wick that are not emitting vaporized fuel reach a combustion temperature and are consumed by the flame. The incineration of the wick limits the exposed length of the wick, thus maintaining a constant burning temperature and rate of fuel consumption.

In contrast, an oil candle or an oil lamp is an object used to produce light using an oil-based fuel source. The use of oil lamps began thousands of years ago and is continued to this day. Oil lamps are a form of lighting, and were used as an alternative to candles before the use of electric lights.

The oil candle uses a combustible liquid, which is drawn up the wick by capillary action and the oil is vaporized and burnt similar to the wax candle. Oil lamps of are used for the particular ambience they produce, or in rituals and religious ceremonies.

Scented wax candles have a scent included in the wax, which is primarily emitted as the wax is heated close to the flame. Scent has also been added to the oil of oil candles; however, the area of exposed heated oil is generally much smaller than that of a wax candle so the scent has less ability to dissipate prior to being combusted in the flame.

Another problem with adding a fragrance to the oil of an oil candle is that it is diluted by the amount of oil in the candle, and if the scent does not easily mix with the oil it may stratify in the oil to further compromise its effectiveness.

What is needed in the art is a dispensing method and apparatus to dispense a scent more effectively than that offered by the prior art.

### SUMMARY OF THE INVENTION

The present invention provides a scent dispersal device closely positioned to the flame of a candle.

The invention in one form is directed to a candle including a wick, a combustible material in contact with a portion of the wick and a scent container. The scent container has an elongated body configured to hold a fragrant liquid. The scent container being configured to generally surround the wick.

The invention in another form is directed to a scent container disperser associated with a candle having a wick and a combustible material in contact with a portion of the wick. The scent container disperser includes an elongated body configured to hold a fragrant liquid. The scent container disperser is configured to generally surround the wick.

An advantage of the present invention is that the fragrance is not diluted in the lamp oil.

Another advantage of the present invention is that the scent container/disperser can be added to virtually any oil candle.

Yet another advantage of the present invention is that the container/disperser can be easily reloaded with fragrance.

### BRIEF DESCRIPTION OF THE DRAWINGS

The above-mentioned and other features and advantages of this invention, and the manner of attaining them, will become more apparent and the invention will be better understood by reference to the following description of an embodiment of the invention taken in conjunction with the accompanying drawings, wherein:

FIG. 1 is a perspective view of an oil candle having an embodiment of scent dispenser of the present invention;

FIG. 2 is a perspective view of the scent dispenser of FIG. 1;

FIG. 3 is a perspective view of the scent dispenser of FIGS. 1 and 2 rotated approximately 90° from the view shown in FIG. 2;

FIG. 4 is an exploded view showing parts of the candle and the associated scent dispenser of FIGS. 1-3;

FIG. 5 is a top illustration of another embodiment of a scent dispenser of the present invention;

FIG. 6 is a perspective view of the scent dispenser of FIG. 5 positioned on an oil candle; and

FIG. 7 is a top view of the scent dispenser of FIGS. 5 and 6.

Corresponding reference characters indicate corresponding parts throughout the several views. The exemplification set out herein illustrates one embodiment of the invention and such exemplification is not to be construed as limiting the scope of the present invention in any manner.

### DETAILED DESCRIPTION OF THE INVENTION

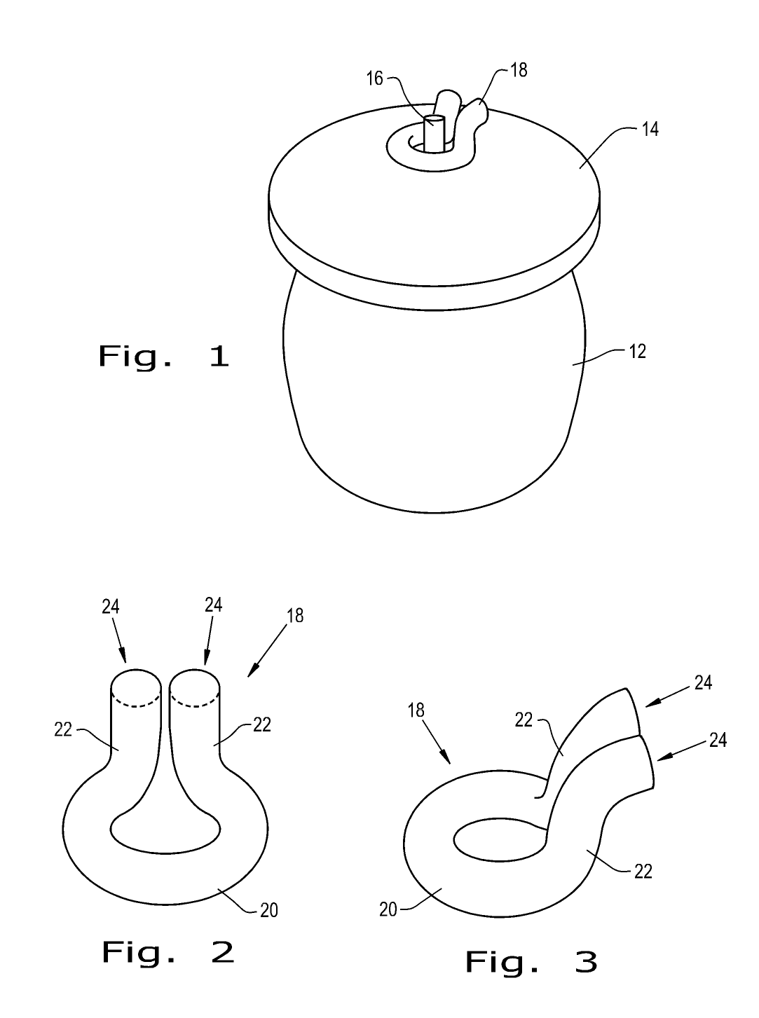

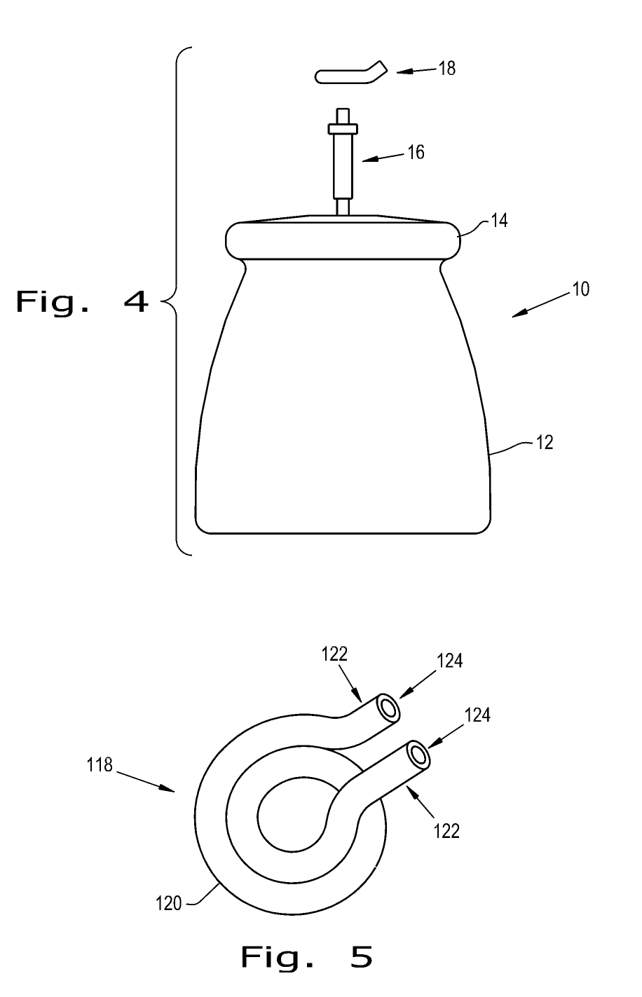

Referring now to the drawings, and more particularly to FIGS. 1-4, there is shown a candle 10 having a containment vessel 12, a lid 14, a wick assembly 16 and a scent holding and dispersal device 18. Scent holding and dispersal device 18 is also referred to as a ScenterRing ™ to create a new mark to refer to the invention in the marketplace. Candle 10 is depicted as an oil candle 10, although other candle and lamps are also contemplated as being used with the present invention. Containment vessel 12 as shown is made of glass and may contain decorations therein. Containment vessel 12 may be made of any material compatible with the combustible fluid used therein. The fluid may be an oil or more specifically a paraffin oil.

Lid 14 provides a convenient top to allow the addition of oil with the removal of lid 14. Lid 14 also has an opening through which wick assembly 16 is placed so that a portion of the wick of wick assembly 16 may extend into the oil to allow the oil to wick up into wick assembly 16 so that candle 10 can be lit in a conventional manner. Wick assembly 16 is a wick holder with a wick extending therethrough.

Scent device 18 is depicted as a piece of glass having a main body 20, elevated portions 22 and openings 24. Scent can be placed with an eye dropper, a syringe or a like device into an opening 24 and scent device 18 placed on candle 10 with a portion of wick assembly 16 extending through the central open space of scent device 18. Heat from a flame burning the oil wicked up wick assembly 16 serves to heat the fragrant material in scent device 18, causing it to disperse and travel into the air to provide a desired scent into the air. Oil candles are beautiful, but they typically do not have a scent. If a scent is added to the oil it simply is not effective. Placing a fragrance scent into the oil of an oil candle of paraffin oil is inefficient in that it does not result in a large throw of the scent. If you put scent inside of the oil candle, the scent will not be very strong. Attempts have been made to overcome this problem. The prior art includes an open reservoir attached to the wick assembly and a ceramic disk having an opening through which the wick is situated with scent being placed on the disk. These prior art method attract dust and insects as well as require cleaning to remove residue.

The present invention advantageously allows the quick installation and the quick change of fragrances. Several scent devices 18 may be prepared or purchased in anticipation of positioning a selected one onto a candle 10. It is contemplated that scent device may or may not be attached to lid 14. Scent device 18 is formed to fit around the glass wick holder, which serves to warm the fragrance oil. The fragrance oil is put inside of the formed glass piece that serves as the reservoir and dispersing device and which causes the scent to permeate the room.

Advantageously the present invention is an improvement over the prior art because prior to this invention, an oil candle could not be scented like a wax or soy candle. An oil candle that uses 99% pure paraffin oil has limited options to use fragrance oil. This invention allows a person to change scent on an oil candle within minutes, using the very same candle. Scent device 18 may or may not be attached to candle 10 and is easy to clean and change the scent.

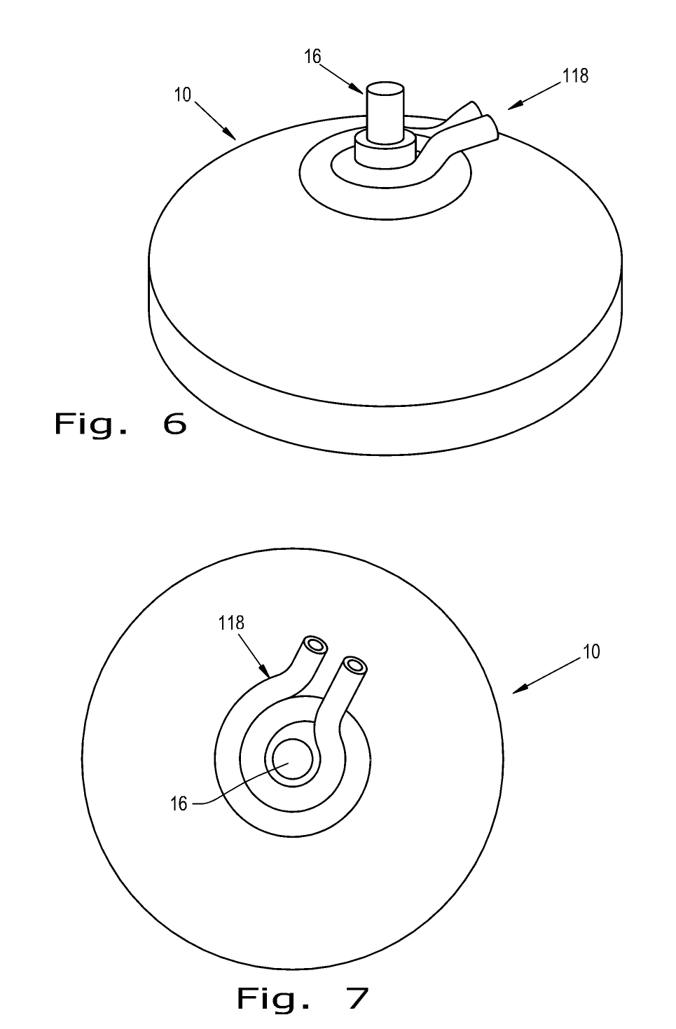

Now, additionally referring to FIGS. 5-7 there is shown another embodiment of the present invention, with some elements having a reference number to which 100 has been added to similar items having a two digit reference number, with the previous description of the elements being applicable to those to which 100 has been added. Scent device 118 here has substantially two complete loops to thereby allow additional fragrance oil to be placed into scent device 118. It is contemplated that openings 24 and 124 can be directed in differing directions and that more or fewer loops of main body 20, 120 may be utilized in making scent device 18, 118. It is further contemplated the one or more openings 24, 124 may be built into scent device 18, 118. The cross sectional profile of the present invention has been illustrated as being circular; however, other cross sectional profiles are also contemplated such as, for example, square or elliptical.

Openings 24 and 124 can vary from each other and may be smaller, larger or the same size as the cross section of main body 20,120. Elevated portions 22 and 122 may have the same cross sectional form as main body 20, 120 or may vary in shape and size.

It is further contemplated that scent devices 18 and 118 may be commercially filled with fragrance oil and that sealed ends of scent devices 18, 118 could be removed by the consumer, or that the sealed ends may melt open upon use to thereby allow convenient storage and use of scent devices 18, 118. It is still further contemplated that scent device 18, 118 may be formed integral with lid 14 and may even be part of the body of lid 14. Scent device 18, 118 may be made of any material compatible with the fragrance oil and which is thermally conductive and preferably flame resistant.

Generally speaking candle 10 has a wick 16 and a combustible material in container 12 that is in contact with a portion of wick 16. The combustible material wicks up wick 16 to be vaporized and burnt by the flame of candle 10. Scent container 18, 118 has an elongated body configured to hold a fragrant liquid, with scent container 18, 118 being configured to generally surround wick 16. The elongated body is hollow as can be clearly seen in FIGS. 2 and 3.

The elongated body has a first end and a second end (ends 24), and the elongated body is hollow from the first end to the second end. Further, the elongated body is a bent hollow tube. Looking at FIG. 2 it can be seen that the elongated body of container/disperser 18 is substantially symmetrical. The first end and the second end of the elongated body are elevated above the mid-section 20. The elongated body may be tubular in nature and may even have a circular cross-section.

As can be seen in the figures the elongated body is circuitous, and may take more than one circuit around wick 16.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A candle, comprising:
a wick;
a combustible material in contact with a portion of said wick; and
a scent container having an elongated body configured to hold a fragrant liquid, said scent container being further configured to generally surround said wick.

2. The candle of claim 1, wherein said elongated body is hollow.

3. The candle of claim 2, wherein said elongated body has a first end and a second end, said elongated body being hollow from said first end to said second end.

4. The candle of claim 1, wherein said elongated body is a bent hollow tube.

5. The candle of claim 4, wherein said elongated body is substantially symmetrical.

6. The candle of claim 5, wherein said elongated body has a first end, a mid-section and a second end, said first end and said second end being elevated above said mid-section.

7. The candle of claim 6, wherein said elongated body is tubular.

8. The candle of claim 7, wherein said elongated body has a circular cross-section.

9. The candle of claim 4, wherein said elongated body is circuitous.

10. The candle of claim 9, wherein said elongated body makes more than one circuit around said wick.

11. A scent container disperser associated with a candle having a wick and a combustible material in contact with a portion of the wick, the scent container disperser including:
an elongated body configured to hold a fragrant liquid, said scent container disperser being configured to generally surround the wick.

12. The scent container disperser of claim 11, wherein said elongated body is hollow.

13. The scent container disperser of claim 12, wherein said elongated body has a first end and a second end, said elongated body being hollow from said first end to said second end.

14. The scent container disperser of claim 11, wherein said elongated body is a bent hollow tube.

15. The scent container disperser of claim 14, wherein said elongated body is substantially symmetrical.

16. The scent container disperser of claim 15, wherein said elongated body has a first end, a mid-section and a second end, said first end and said second end being elevated above said mid-section.

17. The scent container disperser of claim 16, wherein said elongated body is tubular.

18. The scent container disperser of claim 17, wherein said elongated body has a circular cross-section.

19. The scent container disperser of claim 14, wherein said elongated body is circuitous.

20. The scent container disperser of claim 19, wherein said elongated body makes more than one circuit around said wick.
